(19) United States
(12) Patent Application Publication
(10) Pub. No.: US 2026/0175041 A1
(43) Pub. Date: Jun. 25, 2026

(54) TRANSCUTANEOUS SPINAL CORD STIMULATION SYSTEM AND METHODS

(71) Applicant: THE BOARD OF REGENTS OF THE UNIVERSITY OF TEXAS SYSTEM, Austin, TX (US)

(72) Inventors: Yasin Dhaher, Richardson, TX (US); Subaryani Dambawati H Soedirdjo, Dallas, TX (US); Hyungtaek Kim, Seoul (KR)

(73) Assignee: THE BOARD OF REGENTS OF THE UNIVERSITY OF TEXAS SYSTEM, Austin, TX (US)

(21) Appl. No.: 19/427,346

(22) Filed: Dec. 19, 2025

Related U.S. Application Data
(60) Provisional application No. 63/737,436, filed on Dec. 20, 2024.

(51) Int. Cl. A61N 1/40; A61N 1/04

(52) U.S. Cl. CPC A61N 1/40; A61N 1/0456; A61N 1/0476

(57) ABSTRACT

Systems, methods, and devices disclosed herein include a transcutaneous spinal cord stimulation (tSCS) system with a stimulation electrode grid. The stimulation electrode grid includes a plurality of anodes and a plurality of cathodes, and the stimulation electrode grid is operable for placement proximate to a spinous process. The system also includes a particularized electrical field generated by selective activation of the stimulation electrode grid. Moreover, a localized tSCS, provided by the particularized electrical field is operable to cause a neuromodulation effect. Also, the stimulation electrode grid includes a substrate material. The plurality of cathodes and the plurality of anodes are both arranged as rows or columns on the substrate material to form an integrated unit. The stimulation electrode grid includes a plurality of selectively activated electrode array activation configurations, and the neuromodulation effect generated by the system can be used to enhance voluntary extremity function and pain management.

### CROSS REFERENCE TO RELATED APPLICATION

This application claims priority to U.S. Provisional Patent Application Ser. No. 63/737,436, filed Dec. 20, 2024, and titled “TRANSCUTANEOUS SPINAL CORD STIMULATION SYSTEM AND METHODS,” which is incorporated by reference herein in its entirety.

### BACKGROUND

### 1. Field

The present disclosure relates to the field of electrode spinal stimulation.

### 2. Discussion of Related Art

In the past decade, several spinal cord stimulation techniques have demonstrated their effectiveness in neuromodulating spinal circuits, offering potential improvements in voluntary movement for both animals and humans with spinal cord injury (SCI) and stroke. In particular, implanted epidural spinal stimulation (ES) has shown its ability to alter the state of spinal motor circuits, restoring the capacity for independent standing and locomotion, even in individuals with motor-complete SCI when paired with locomotor training. However, these implanted stimulation techniques are invasive and involve expensive surgical procedures for electrode placement.

It is with these observations in mind, among others, that various aspects of the present disclosure were conceived and developed.

### SUMMARY

Systems, methods, and devices disclosed herein can address the aforementioned issues. For instance, a system of transcutaneous spinal cord stimulation can include a current generator and a stimulation electrode grid including a plurality of anodes and a plurality of cathodes. The stimulation electrode grid can be operable for placement proximate to a spinous process. Additionally, a particularized electrical field can be generated by selectively activating the stimulation electrode grid. Furthermore, the system can include a localized transcutaneous spinal cord stimulation (tSCS), provided by the particularized electrical field, and operable to cause a neuromodulation effect transcutaneously, thus avoiding the complications of an invasive surgical procedure.

In some examples, the stimulation electrode grid can include a substrate material. Additionally, the plurality of cathodes and the plurality of anodes can both be arranged as rows or columns on the substrate material to form an integrated unit, with the anodes and cathodes arranged next to each other on the integrated unit. The stimulation electrode grid can also include an electrode array activation configuration with the plurality of anodes forming a first outer column of electrodes and a second outer column of electrodes, and/or the plurality of cathodes forming a first inner column of electrodes and a second inner column of electrodes. Moreover, the stimulation electrode grid can include at least a four-by-four electrode array with the plurality of anodes including four electrodes located in a first quadrant (e.g., quadrant I) of the four-by-four electrode array, and the plurality of cathodes including four electrodes located in a third quadrant (e.g., quadrant III) of the four-by-four electrode array.

In some instances, the stimulation electrode grid can include an electrode array activation configuration with the plurality of anodes forming a first outer column of electrodes and a first inner column of electrodes, and/or the plurality of cathodes forming a second outer column of electrodes and a second inner column of electrodes. Additionally, or alternatively, the stimulation electrode grid can include an electrode array activation configuration with the plurality of anodes forming a first two-by-two electrode array, and/or the plurality of cathodes forming a second two-by-two electrode array adjacent to the first two-by-two electrode array. Moreover, the stimulation electrode grid can include an electrode array rotated, for instance, 45° relative to a longitudinal spinal axis to provide a diagonal stimulation montage. The electrode array can be rotated another amount, such as 25°, 30°, 35°, 40°, 50°, 55°, 60°, and so forth. Furthermore, the stimulation electrode grid can be operable for placement with a midpoint between a T10-T11 spinous process and/or at other places along the spine. The system can also include a mobile computing device communicatively coupled to the stimulation electrode grid, and the selective activation can be in response to a user input at a graphical user interface of the mobile computing device.

In some scenarios, a tSCS device can include a substrate material and/or a stimulation electrode grid disposed on the substrate material. The stimulation electrode grid can include a plurality of anodes and a plurality of cathodes, and can be operable for placement proximate to a spinous process. The tSCS device can also include a power supply, to provide power to its various components. Furthermore, a current generator can be operable to provide a current signal to the stimulation electrode grid resulting in generation of a particularized electrical field. The particularized electrical field can be used to provide localized tSCS.

In some examples, the stimulation electrode grid can include a four-by-four electrode array selectable between a plurality of electrode array activation configurations to provide a plurality of different particularized electrical fields. The plurality of electrode array activation configurations can be selectively activated and can include a first electrode array activation configuration with outer columns of electrodes activated to be the plurality of anodes and inner columns of electrodes activated to be the plurality of cathodes, and/or a second electrode array activation configuration with electrodes at a first side activated to be the plurality of anodes and electrodes at a second side, opposite the first side, activated to be the plurality of cathodes. Furthermore, the plurality of electrode array activation configurations can include a first electrode array activation configuration with electrodes at a first quadrant activated to be the plurality of anodes and electrodes at a second quadrant activated to be the plurality of cathodes, and/or a second electrode array activation configuration with electrodes at a first side activated to be the plurality of anodes and electrodes at a second side, opposite the first side, activated to be the plurality of cathodes.

In some instances, the plurality of electrode array activation configurations can include an electrode array activation configuration with a first two-by-two array of electrodes activated to be the plurality of anodes and a second two-by-two array of electrodes, adjacent to the first two-by-two array, activated to be the plurality of cathodes. Additionally, the electrode array activation configuration can include a first row of inactive electrodes above the plurality of anodes and the plurality of cathodes, and/or a second row of inactive electrodes below the plurality of anodes and the plurality of cathodes. The localized tSCS can provide a neuromodulation effect on motion and/or pain reflex pathways. In this way, the disclosed technology can be used for pain management, for instance, by inhibiting a pain pathway of the spinal cord, which can correspond to a motion and/or a reflex pathway-Furthermore, the particularized electrical field can target one or more lumbar spinal circuits which innervate a lower and/or upper limb muscle, such as a tibialis anterior muscle, and/or inhibits a pain pathway. The stimulation electrode grid can include a grid layout with an equal distance between electrodes, and/or a number of cathodes equaling a number of anodes. Moreover, the device can include a positioning guide operable to assist in placement of the stimulation electrode grid. Also, the device can include a plurality of predetermined stimulation modes stored in a memory of a computing device communicatively coupled to the stimulation electrode grid. The plurality of predetermined stimulation modes can correspond to different target diseases.

In some scenarios, a method of transcutaneous spinal cord stimulation can include selecting at tSCS procedure. Furthermore, the method can include positioning a stimulation electrode grid proximate to a spinous process. The method can also include selectively activating electrodes of the stimulation electrode grid to generate a particularized electrical field based on a selective activation which assigns first electrodes as anodes and a selective activation which assigns second electrodes as cathodes. Additionally, the method can include providing, using the particularized electrical field, localized tSCS.

In some examples, the localized tSCS can include a plurality of 0.1 ms biphasic pulses delivered at 100 Hz, and/or the localized tSCS can use 40 mA distributed across eight channels corresponding to eight electrodes, in parallel, resulting in a delivery of 5 mA per channel. Additionally, the selective activating of the electrodes can include receiving a user input at a graphical user interface of a mobile device associated with a user, the user input selecting an electrode grid activation configuration from a plurality of electrode grid activation configurations presented at the graphical user interface. The localized transcutaneous spinal cord stimulation can modulate a spinal reflex excitability corresponding to extremities of a treatment recipient. Furthermore, the method can include providing data representing the selective activation to a machine-learning model trained to identify patterns of use of different selective activations.

### BRIEF DESCRIPTION OF THE FIGURES

FIG. 1A illustrates an example system including placement of a stimulation electrode grid device proximate to a spinous process for performing a transcutaneous spinal cord stimulation procedure.

FIG. 1B illustrates an example system including a stimulation electrode grid device including a plurality of electrodes on a substrate for performing a transcutaneous spinal cord stimulation procedure.

FIG. 1C illustrates an example system including one or more selectable electrode array activation configurations for performing a transcutaneous spinal cord stimulation procedure.

FIG. 2 illustrates an example system for performing a transcutaneous spinal cord stimulation procedure with one or more computing devices of a treatment platform.

FIG. 3 illustrates an example method of performing a transcutaneous spinal cord stimulation procedure.

### DETAILED DESCRIPTION

It will be appreciated that numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the examples described herein. The drawings are not necessarily to scale and the proportions of certain parts may be exaggerated to better illustrate details and features of the present disclosure.

The systems, methods, and devices disclosed herein can use non-invasive spinal stimulation to modulate spinal excitability, for instance, by non-invasive transcutaneous spinal cord stimulation (tSCS), which can be used to treat a broad range of patients without requiring surgical procedures. These systems can enhance voluntary lower extremity function, including standing and locomotion, and can reduce spasticity, for example, by pairing tSCS with rehabilitation training. One factor for tSCS efficacy is the spatial extent of the induced electric field. Some other systems position an electrode over the intended spinal cord target region for modulation, while the return electrode is placed in a functionally distant area. Computational modeling studies indicate that employing this bipolar scheme results in a diffused electric field, not directly underneath the electrode segment of interest, making the mechanistic interpretation of any functional gains challenging. In-silico examinations, performed by computational modeling studies, can indicate that some tSCS studies may not have delivered the maximum or optimal electric field to the intended target. The bipolar tSCS schemes can lack spatial precision.

Accordingly, the systems disclosed herein address several challenges for effective implementation of non-invasive spinal stimulation into rehabilitation programs, including establishing targeted stimulation paradigms.

For example, the systems disclosed herein can use a localized electrical field to affect various mechanisms involving spinal pathways, including activation of dorsal root afferents, modifications in motor commands transmitted along the corticospinal pathway, and/or stimulation-induced modulation in the excitability of propriospinal neurons networks and spinal interneurons. Other mechanisms may also contribute to the effect of spinal stimulation by using the system(s) disclosed herein, including changes in polysynaptic spinal reflexes and engaging both motoneurons and interneurons. The tSCS techniques disclosed herein can engage sensory-motor pathways, including intraneuronal circuits, during stimulation, which can be confirmed with human studies.

Because the sensory-motor pathways of the spinal cord correspond to pain sensation pathways, the systems disclosed herein can be used for a variety of pain management scenarios. These can include treatment for injuries, nerve pressure points and/or nerve damage, medical conditions, infections, and/or other causes of pain or chronic pain. Furthermore, the techniques disclosed herein can be used to treat phantom pain associated with amputation, myelopathy, clonus, and/or spasticity associated with nerve damage, which can have many causes, such as a stroke. The techniques disclosed herein can have additional benefits over other techniques due to plasticity changes that may occur following treatment. Moreover, the systems disclosed herein can be customized (e.g., by using different electrode configuration activations, and by using aggregated data from a cloud based platform) to match a particular patient, which can address variations in patient anatomy from one patient to another.

In some instances, the disclosed technology can be applied to the lumbar spinal cord in neurologically intact participants. The system can include an electrode grid which delivers less spatially diffused stimulation, unlike other cathode/anode pair stimulation paradigms where electrodes are placed on different parts of the participant's body (e.g., shoulder, abdomen, back, and pelvis). By locating the cathode and anode in close proximity, the spatial resolution of the stimulation can be increased while ensuring that the maximum intensity of the electric field remains above a predetermined threshold (e.g., 0.15 V/m) for effective neuromodulation. In addition, the system can employ a stimulation paradigm with two or more montages that generate a net-applied electric field in the transverse and/or diagonal directions. In some scenarios, these arrangements of anode and cathode can be selected as “feasibility/proof of concept” montages, with the model indicating that they lead to discernible variations in both the direction and magnitude of the electric field within the gray matter.

In some examples, the system(s) disclosed herein can be used for targeted neuromodulation. For example, the stimulation electrode grid can provide localized and targeted stimulation, which can provide more effective modulation of specific spinal circuits and pain pathways more effectively. Moreover, the system(s) can result in personalized treatment in that the stimulation electrode grid can provide clinicians a way to customize the stimulation to match each patient's unique spinal anatomy and/or pain/reflex responses to stimulation. This approach can enhance pain therapies like transcutaneous electrical nerve stimulation (TENS), which may otherwise lack precise control over the electric field's interaction with pain targets. The disclosed system(s) can also result in improved efficiency and adaptivity, as compared to other techniques which require significant time and effort to identify the treatment configuration. The system(s) disclosed herein can be more efficient and provide an easier setup.

In some scenarios, the system(s) disclosed herein can be implemented as a pain reliever system. For instance, this technology can be used to reduce broad, uncontrolled stimulation by providing more targeted neuromodulation using the stimulation electrode grid which includes both cathodes and anodes on a single, integrated unit. This can avoid the broad, non-specific stimulation of other tSCS and TENS platforms which can lead to unintended side effects. The more focused approach disclosed herein can lead to improved pain management by reducing unnecessary activation of non-pain related pathways. Moreover, the disclosed technology can include a more flexible electrode placement procedure. The disclosed stimulation electrode grid design can make it easier for clinicians to place electrodes in a way that matches the patient's anatomy, which can reduce the difficulty of providing effective stimulation.

In some instances, the disclosed technology can be integrated into various products and/or services which use a stimulation electrode grid-based system. For example, neuromodulation technology can include the disclosed grid configurations of anode-cathode pairs to generate one or more spatially distinct electric field(s). This can result in customizable stimulation profiles such that users can configure which electrodes are active, through selective electrode activation, which can provide control over the electric field's shape and direction. As such, targeted neuromodulation in which localized electric fields can be generated in specific spinal segments of interest can be adapted to individual patients' needs, which can result in personalized treatment options.

Additional advantages of the systems, methods, and devices discussed herein will become apparent from the detailed description below.

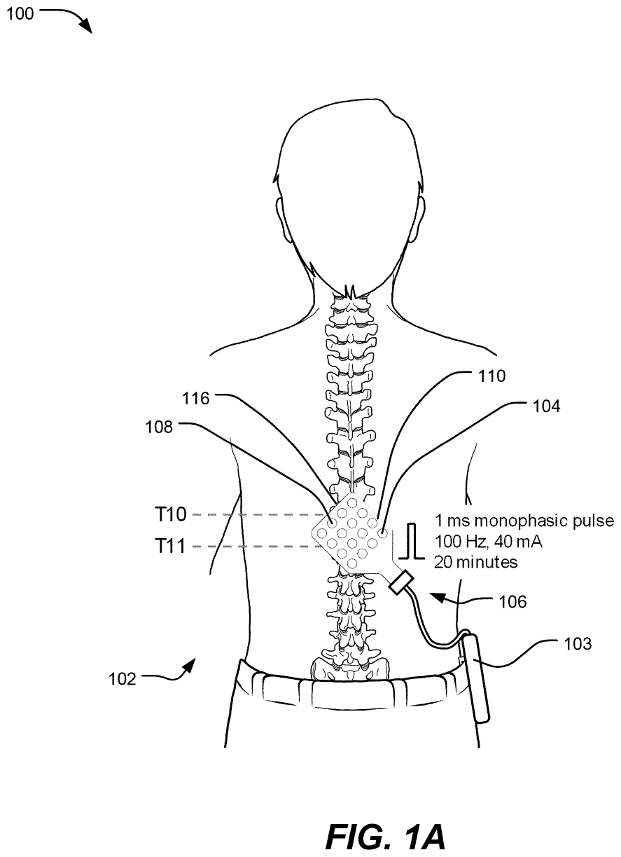

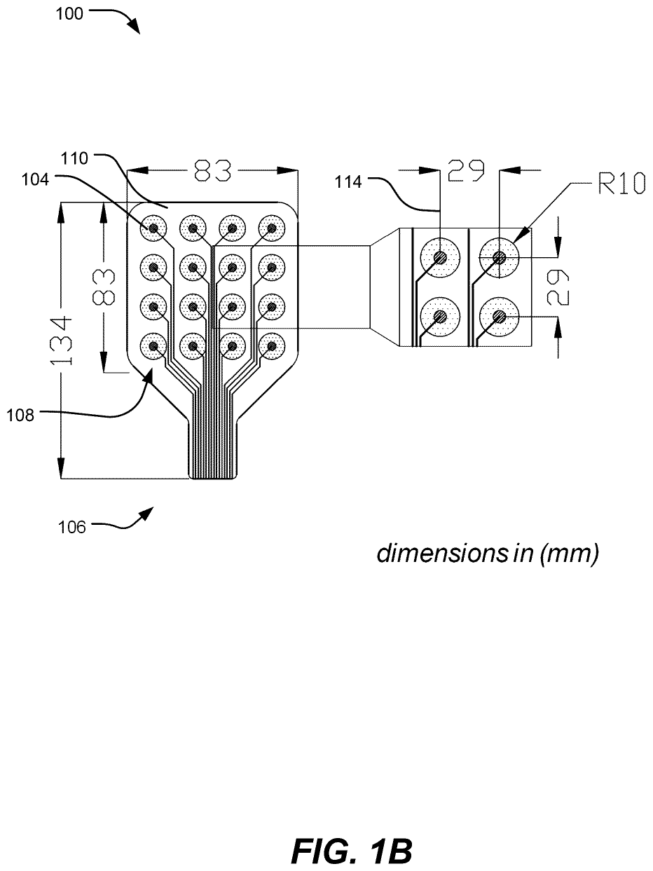

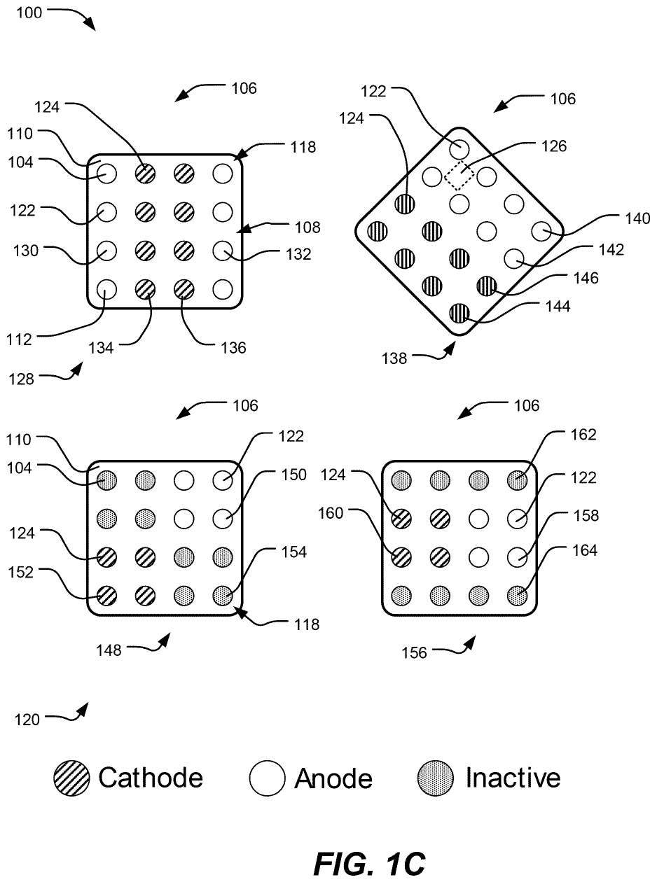

FIGS. 1A-1C illustrate an example system 100 for performing a tSCS procedure 102.

In some examples, the stimulation electrode grid design can provide for custom stimulation montages. For instance, the tSCS procedure 102 can be used to create a distinct electric field, that can be used for disease-specific and/or subject-specific neuromodulation. In TENS application (e.g., lower-back pain management), electrodes 104 can be applied at or around the specific targets. The distinct electric field can differentially modulate tibialis anterior (TA) flexion reflex, which is a reflex synonymous to withdrawal reflex (e.g., pain mediated). As such, the disclosed tSCS procedure 102 can improve the precision and effectiveness of TENS treatments by better controlling pain pathways. Moreover, treatment using the tSCS procedure 102 may be linked to improved human locomotion, such as improved clinical outcomes (e.g., walking).

In some examples, the tSCS procedure 102 can be performed with a tSCS device 106 including a stimulation electrode grid 108 formed by the plurality of electrodes 104 disposed on a substrate material 110. For instance, the plurality of electrodes 104 can include one or more rows and/or columns 112 of electrodes 104. The plurality of electrodes 104 can be spaced apart by a predetermined spacing distance 114 to provide the localized electrical field 116. Furthermore, the plurality of electrodes 104 can be arranged on the substrate material 110 to form an electrode array, such as a four-by-four electrode array 118, a three-by-eight electrode array, and/or electrode arrays of other dimensions. The plurality of electrodes 104 can be selectively activated to select between a plurality of different electrode grid activation configurations 120, with different electrodes 104 designated as anodes 122 and cathodes 124. Both of the plurality of anodes 122 and the plurality of cathodes 124 can be disposed next to each other and/or in close proximity to each other on the substrate material 110 to form the tSCS device 106 as an integrated unit.

In some scenarios, the substrate material 110 can be formed of a biocompatible material that facilitates prolonged adhesion to the skin surface for a duration of multiple days without significant loss of conductivity or attachment integrity. The material can exhibit properties including flexibility, moisture resistance, and/or hypoallergenic characteristics, thereby providing sustained user comfort and functionality over an extended period of time.

In some examples, the system 100 can include a positioning guide 126 operable to assist in placement of the stimulation electrode grid 108. For instance, the positioning guide 126 can be a feature of the tSCS device 106 which aids in placement orientation, such as an embedded accelerometer sensor or similar technology incorporated into the stimulation electrode grid 108 to analyze its orientation. Furthermore, the positioning guide 126 can include an interface on a mobile device, for instance, using a camera of the mobile device to analyze and/or provide feedback for a placement of the stimulation electrode grid. Additionally, the stimulation electrode grid 108 can include visual and/or tactile markers to indicate orientation.

In some instances, the tSCS procedure 102 can be administered using a grid of electrodes 104 (e.g., the stimulation electrode grid 108) and a current generator 103 (e.g., a constant-current generator). The grid of stimulating electrodes 104 can be fabricated using pre-gelled electrodes with a diameter of 0.5 inches, and can comprise eight pairs of anodes 122 and cathodes 124. The T10 spinous process can be identified by palpation, and the central point of the stimulation electrode grid 108 can be aligned with the midpoint between the T10-T11 spinous process. The stimulation electrode grid 108 can cover the vertebral levels T10 to T12, which correspond to the L1 to L5 cord segments. These segments can correspond to the segmental innervation of the TA muscle, from which the effect of the tSCS procedure 102 can be assessed.

In some examples, a stimulation protocol of the tSCS procedure 102 can include 0.1-ms biphasic pulses delivered at 100 Hz. A total of 40 mA can be distributed across eight channels in parallel (e.g., corresponding to eight anode-cathode pairs), resulting in a delivery of 5 mA per channel. The total current can be based on results of a high-fidelity finite element model, which can indicate that the 40 mA current is sufficient to modulate firing patterns of axons within ascending and descending white matter tracts and neurons in the spinal gray matter as well for the two selected montages. The intensity of stimulation can be gradually increased from 2 to 40 mA over 1 minute and can be maintained at 40 mA for 20 minutes. A patient receiving treatment can be seated during the tSCS procedure 102.

In some instances, the tSCS procedure 102 can implement one or more selectable electrode array activation configuration(s) 120. For instance, a first electrode array activation configuration 128 can include the plurality of anodes 122 forming a first outer column 130 of electrodes 104 and a second outer column 132 of electrodes 104. The first electrode array activation configuration 128 can also include the plurality of cathodes 124 forming a first inner column 134 of electrodes 104 and a second inner column 136 of electrodes 104. Additionally, the first electrode array activation 128 can include an electrode arrangement for transversal grid stimulation in that the first outer column 130, the second outer column 132, the first inner column 134, and/or the second inner column 136 can run parallel to a longitudinal axis of the spine.

Moreover, the selectable electrode array activation configurations 120 can include a second electrode array activation configuration 138 with the plurality of anodes 122 forming a first outer column 140 of electrodes 104 and a first inner column 142 of electrodes 104. The second electrode array activation configuration 138 can also include the plurality of cathodes 124 forming a second outer column 144 of electrodes 104 and a second inner column 146 of electrodes 104. In some scenarios, the second electrode array activation configuration 138 (e.g., and/or any of the electrode array activation configurations 142 disclosed herein) can be rotated 45° relative to the longitudinal spinal axis, or any other angle, to provide diagonal grid stimulation.

Additionally, the selectable electrode array activation configurations 120 can include a third electrode array activation configuration 148 with the four-by-four electrode array 118. The four-by-four electrode array 118 can include the plurality of anodes 122 being four electrodes located in a quadrant | 150 of the four-by-four electrode array 118; and/or the plurality of cathodes 124 being four electrodes located in a quadrant III 152 of the four-by-four electrode array 118. In this way, the plurality of anodes 122 can be located in an opposite quadrant (e.g., a diagonal quadrant) from the plurality of cathodes 124. Additionally or alternatively, the plurality of cathodes 124 can be located in a quadrant II 154 of the four-by-four electrode array 118, such that the plurality of cathodes 124 are in an adjacent quadrant to the plurality of anodes 122.

Furthermore, the selectable electrode array activation configurations 142 can include a fourth electrode array activation configuration 156 with the plurality of anodes 122 forming a first two-by-two electrode array 158, and/or the plurality of cathodes 124 forming a second two-by-two electrode array 160 adjacent to the first two-by-two electrode array 158. In the fourth electrode array activation configuration 156, a first row of electrodes 162 above the anodes 122 and the cathodes 124 can be an inactive row of electrodes 104, and/or a second row of electrodes 164 below the anodes 122 and the cathodes 124 can be an inactive row of electrodes 104.

Any of the selectable electrode array activation configurations 120 discussed herein can be combined together and/or selectably toggled between each other. The various selectable electrode array activation configurations 120 disclosed herein can be used to generate custom electrode montages for focused and/or localized stimulation in a multi-configurable manner using a single device. In some examples, the input current passing through each electrode 104 of the stimulation electrode grid 108 can be equal in amplitude, shape, and/or pattern. For example, the stimulation current profile(s) can include continuous 0.1 ms biphasic pulses at 100 Hz, and/or with 5 mA per channel. The system 100 can operate with an initial current ramp up and/or an end-of-treatment ramp down at a rate of 0.625 mA per channel per second. Furthermore, the system 100 can include an impedance monitor for each channel of the stimulation electrode grid 108 which can detect poor electrode-to-skin contact and/or notify the user to provide a safe tSCS procedure 102.

In some instances, the four-by-four electrode array 118 can include circular electrodes having a 0.5 inch diameter and/or an inter-electrode spacing distance of 0.7 inches. The distance between each of the electrodes of the four-by-four electrode array 118 can be equal. Furthermore, the substrate material 110 on which the stimulation electrode grid 108 is fabricated can be a disposable material, a semi-disposable material, a biodegradable material, a biocompatible material, combinations thereof, and/or the stimulation electrode grid can be embedded into a stretchable material. Additionally, the grid arrangement can be non-square, such as a circular arrangement, a honeycomb arrangement, or a triangular arrangement.

In some scenarios, the system 100 can include a power supply voltage source which can be a battery-powered current generator 103 with multiple output channels (e.g., corresponding to the multiple electrodes). The battery can be a rechargeable battery or a single use battery. Additionally, the output channels can include eight anode channels and/or eight cathode channels.

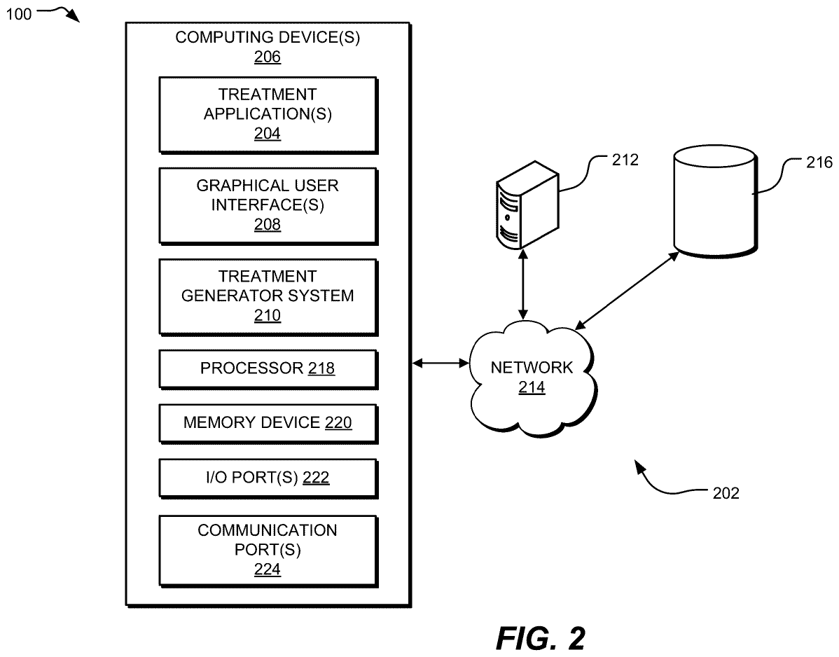

FIG. 2 depicts an example system 100 for performing the tSCS procedure 102 using a treatment platform 202 including one or more treatment application(s) 204. The system 100 depicted in FIG. 2 can be similar to, identical to, and/or can form at least a portion of the system 100 depicted in FIGS. 1A-1C.

In some examples, the tSCS procedure 102 can be performed using the treatment platform 202, which can include a telehealth system implemented as one or more treatment applications 204 operating at one or more computing device(s) 206. The computing device(s) 206 (e.g., a mobile device) can be associated with a patient/user receiving treatment from the tSCS procedure 102, for instance, at their home to self-administer the treatment. For instance, the computing device 206 can establish a wireless connection with the stimulation electrode grid 108 such that a user can control operation of the stimulation electrode grid 108 with the computing device(s) 206.

In some instances, the computing device(s) 206 can include a computer, a personal computer, a desktop computer, a laptop computer, a terminal, a workstation, a cellular or mobile phone, a mobile device, a smart mobile device, a tablet, a wearable device (e.g., a smart watch, smart glasses, a smart epidermal device, etc.), an Internet-of-Things (IoT) device, a smart home device, a virtual reality (VR) device, an augmented reality (AR) device, the tSCS device 106, and/or the like. It will be appreciated that specific implementations of these devices may be of differing possible specific computing architectures. Also, the computing device 206 can be communicatively coupled to a current generator 103 with a plurality of channel outputs, as discussed above.

In some scenarios, the computing device 206 can include a graphical user interface (GUI) 208. The GUI 208 can be used by the user to select a type of tSCS procedure 102 and/or various parameters of the tSCS procedure 102. For instance, the GUI 208 can present indicators (e.g., visual icons) which, upon receiving a user input, cause the stimulation electrode grid 108 to output the different selectable electrode array activation configurations 120. The user can provide input to the GUI 208 to select the first electrode array activation configuration 128, the second electrode array activation configuration 138, the third electrode array activation configuration 148, the fourth electrode array activation configuration 156, and/or any combination thereof. Additionally, the GUI 208 can present options to select and/or adjust different treatment parameters, such as an electrode pairing input which selects and/or creates a particular anode-cathode pair (e.g., a customized montage), a duration input to determine a duration of the stimulation current at one or more anode-cathode pairs, a pulse timing input to determine a spacing time between one or more stimulation pulses, and/or an amplitude input to determine a stimulation intensity or an increasing and/or decreasing intensity profile for a plurality of stimulation pulses.

Furthermore, the treatment platform 202 can include a treatment generator system 210 which determines one or more of the selectable electrode array activation configurations 120 to be presented at the GUI 208. For example, a remote service (e.g., a cloud service) and/or a local application operating at the computing device 206 can aggregate selection data from one or more users, such as a plurality of users. Over time, the selection data, indicating which configurations were selected by the users, can undergo various statistical analysis (e.g., using one or more trained machine-learning models) to detect patterns in which types of tSCS procedures 102 are being selected by the users, which tSCS procedures 102 result in a high level of satisfaction, and/or which treatment parameters are being selected at a higher rate than other treatment parameters by the users. Moreover, the treatment generator system 210 can detect which tSCS procedures 102 are being used for which symptoms and/or diseases, and an effectiveness of the selected tSCS procedures for the particular symptoms and/or diseases. Once identified, the treatment generator system 210 can cause high-frequency and/or more popular treatments or parameters to be presented at the GUI 208 as a selectable option by the user. In this way, treatment configurations and parameters that are having a higher success rate with some user can be identified and provided as an option to other users. The treatment platform 202 can provide updates to the treatment applications 204 (e.g., at a mobile application interface) to add AI-informed stimulation patterns to a list of patient-selectable stimulation patterns.

Additionally, the treatment platform 202 can receive user inputs indicating symptoms of the user and, using the trained machine-learning model(s), can provide suggestions for personalized treatment plans and/or patient-specific grid configurations based on the symptoms data. For example, the user can provide patient-specific information, such as symptoms data and/or medical history data, through the mobile application interface. The treatment platform 202 can use the stored personal treatment history to recommend an optimal grid layout and/or an optimal polarity configuration tailored to that particular user.

In some instances, the configuration of electrode polarity, such as assignments of a cathode status or an anode status for the electrodes 104, can be set through the computing device 206 (e.g., one or more inputs at the GUI 208 of mobile device). For example, the system 100 can be integrated with the mobile application interface, which can monitor the progress as the user performs treatment routines. As noted above, the mobile application interface can present selectable options for a user to select and/or customize the timing and/or duration of the treatment and/or stimulation sessions. Furthermore, the mobile application interface can include the positioning guide 126 to assist in placement of the stimulation electrode grid 108, for instance, using augmented reality overlays onto the camera images. This can include a detection module which determines an orientation of the stimulation electrode grid 108 and/or generates adjustment recommendations. Moreover, the mobile application interface can detect whether one of the channels becomes disconnected and, in response, can generate a notification to be presented at the GUI 208.

In some examples, the system 100 can include a remote and/or cloud service. A plurality of predetermined (e.g., pre-set) stimulation modes, which correspond to specific target diseases (e.g., lower limb motor functionality, back pain, phantom pain, and the like), can be stored at a memory storage device 220, at the computing device 206 (e.g., at the mobile device, the cloud server, at the battery-powered current generator 103, and/or combinations thereof). In some scenarios, the plurality of predetermined stimulation modes can be integrated into a mobile application (e.g., one of the treatment application(s) 204). The system 100 can also store and/or analyze personal treatment history associated with a particular user. Furthermore, the system 100 can store de-identified physiological data in a secure environment, such as a secure remote server.

In some instances, the systems 100 for performing the tSCS procedure 102 can be integrated with multiple, different types of sensors, such as an electroencephalogram (EEG) sensor, an EMG sensor, a smart watch, and/or combinations thereof. Additionally, the system 100 can detect unusual physiological activities (e.g., heart rate, via motion sensor data, via EMG data, etc.) by using local and/or remote monitoring of the treatment platform 202, and can use an alert system to improve safety. The alerts can be provided to the user and/or a service provider. Furthermore, the system 100 can detect unusual usage patterns such as extended treatment duration and/or a high current amplitude. Also, the system 100 can monitor and/or adjust the treatment protocols remotely (e.g., using the cloud server). These adjustable treatment protocols can include configurations of the electrode polarities, current intensity, current pattern, and or the shape of the stimulation current. The system can also be implemented as a system-on-chip (SoC), wherein all or a portion of the components, such as the processor 218, memory device 220, and/or functional modules, are integrated onto a single semiconductor substrate. The SoC implementation may use a combination of hardware, firmware, and/or software to provide the functionalities disclosed herein. The SoC may be fabricated using complementary metal-oxide-semiconductor (CMOS) technology or any other suitable semiconductor fabrication technology.

In some examples, the computing device 206 can be an additional separate device from the tSCS device 106, and/or the computing device 206 can be formed into the tSCS device 106. The tSCS device 106, with the stimulation electrode grid 108, can receive one or more control transmissions from the computing device 206 and/or can transmit the data generated by the tSCS device 106 to one or more computing device 206. These transmissions can be sent directly between the tSCS device 106 and the computing device 206 (e.g., via Bluetooth™ or Wi-Fi), and/or by using at least one server device 212 (e.g., using a cloud service). The computing device 206 can receive and/or transmit using a network 214 (e.g., using one or more network connections). The network 214 can include any type of network, such as the internet, an intranet, a Virtual Private Network (VPN), a Voice over Internet Protocol (VoIP) network, a wireless network (e.g., Bluetooth™), a cellular network (e.g., 4G, 5G, LTE, etc.), satellite, combinations thereof, etc. The network 214 can include communications network(s) with numerous components such as, but not limited to gateways routers, server(s) 212, and registrars, which enable communication across the network 214. In one implementation, the network(s) 214 can include multiple ingress/egress routers, which may have one or more ports, in communication with the network 214. Additionally, or alternatively, the treatment platform 202, the computing device(s) 206, the server(s) 212, and/or the tSCS device 106 can access and be accessed by the network 214 via another type of communications network, which may be a public switched telephone network (PSTN) operated by a local exchange carrier (LEC) and/or a wireless network.

In some instances, the at least one server 212 can host a website or application of the treatment platform 202 (e.g., the treatment application(s) 204), which the computing device(s) 206 and/or the tSCS device 106 may visit to access the treatment platform 202, provide inputs to the treatment platform 202, and/or to control the stimulation electrode grid 108. To perform these operations the server 212 can access (e.g., read and/or write) one or more database(s) 216. The website or application can receive the inputs and can analyze the inputs to generate outputs for the treatment platform 202 (e.g., using the treatment generator system 210) which can be stored at the database(s) 216. The server 212 may be a single server, a plurality of servers with each such server being a physical server or a virtual machine, or a collection of both physical servers and virtual machines. In another implementation, a cloud service hosts one or more components of the treatment platform 202. The server 212 may represent an instance among large instances of application servers in a cloud computing environment, a data center, or other computing environment. The server 212 can access the data stored at the one or more database(s) 216. The treatment platform 202, tSCS device 106, the computing device(s) 206, and/or other resources connected to the network 214 may access one or more other servers to access one or more websites, applications, web services interfaces, storage devices, computing devices, or the like, thus providing the treatment platform 202.

The computing device 206 may be a computing system capable of executing a computer program product to execute a computer process. Data and program files may be input to the computing device 206, which reads the files and executes the programs therein. Some of the elements of the computing device 206 can include one or more hardware processors 218, one or more memory devices 220, and/or one or more ports, such as input/output (IO) port(s) 222 and communication port(s) 224. Various elements of the computing device 206 may communicate with one another by way of the communication port(s) 224 and/or one or more communication buses, point-to-point communication paths, or other communication means.

The processor 218 may include, for example, a central processing unit (CPU), a microprocessor, a microcontroller, a digital signal processor (DSP), a SoC, a graphics processing unit (GPU), and/or one or more internal levels of cache. There may be one or more processors 218, such that the processor 218 comprises a single central-processing unit, or a plurality of processing units capable of executing instructions and performing operations in parallel with each other, referred to as a parallel processing environment.

The computing device 206 may be a single computer, a plurality of computers (e.g., a distributed computer), or another type of computer, such as one or more external computers made available via the cloud computing architecture. The presently described technology is optionally implemented in software stored on a data storage device(s) such as the memory device(s) 220 (e.g., locally stored at the computing device 206), and/or communicated via one or more of the ports 222 or 224 to the treatment platform 202, thereby transforming the computing device 206 into a special purpose machine for implementing the operations of the tSCS procedure 102 described herein.

The one or more memory device(s) 220 may include any non-volatile data storage device capable of storing data generated or employed within the computing device 206, such as computer executable instructions for performing a computer process, which may include instructions of both application programs and an operating system (OS) that manages the various components of the computing device 206. The memory device(s) 220 may include magnetic disk drives, optical disk drives, solid state drives (SSDs), flash drives, and the like. The memory device(s) 220 may include removable data storage media, non-removable data storage media, and/or external storage devices made available via a wired or wireless network with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, SSDs, and the like. The one or more memory device(s) 220 may include volatile memory (e.g., dynamic random-access memory (DRAM), static random-access memory (SRAM), etc.) and/or non-volatile memory (e.g., read-only memory (ROM), flash memory, etc.).

Computer program products containing mechanisms to effectuate the systems and methods in accordance with the presently described technology may reside in the memory device(s) 220 which may be referred to as machine-readable media. It will be appreciated that machine-readable media may include tangible non-transitory medium capable of storing or encoding instructions to perform operations of the treatment platform 202. The machine-readable media can store computer-readable instructions for execution by a machine, and/or can be capable of storing or encoding data structures and/or modules utilized by or associated with such instructions.

In some implementations, the computing device 206 includes one or more ports, such as the I/O port 222 and the communication port 224, for communicating with other computing, network, or devices. It will be appreciated that the I/O port 222 and the communication port 224 may be combined or separate and that more or fewer ports may be included in the computing device 206.

The I/O port 222 may be connected to an I/O device, or other device, by which information is input to or output from the computing device 206. For instance, input devices can convert a human-generated signal, such as, human voice, physical movement, physical touch or pressure, and/or the like, into electrical signals as input data into the computing device 206 via the I/O port 222. Similarly, output devices may convert electrical signals received from the computing device 206 via the I/O port 222 into signals that may be sensed as output by a human, such as sound, light, and/or touch. The input device may be an alphanumeric input device, including alphanumeric and other keys for communicating information and/or command selections to the processor 218 via the I/O port 222. The input device may be another type of user input device including, but not limited to: direction and selection control devices, such as a mouse, a trackball, cursor direction keys, a joystick, and/or a wheel; one or more sensors, such as a camera, a microphone, a positional sensor, an orientation sensor, an inertial sensor, an accelerometer; and/or a touch-sensitive display screen (“touchscreen”). The output devices may include, without limitation, a display, a touchscreen, a speaker, a tactile or haptic output device, and/or the like. In some implementations, the input device and the output device may be the same device, for example, in the case of a touchscreen.

In one implementation, the communication port 224 is connected to the network 214, and the computing device 206 may receive network data useful in executing the methods and systems set out herein as well as transmitting information and network configuration changes determined thereby. Stated differently, the communication port 224 can connect the computing device 206 to one or more communication interface devices configured to transmit and/or receive information between the computing device 206 and other devices by way of one or more wired or wireless communication networks or connections. For instance, one or more such communication interface devices may be utilized via the communication port 224 to communicate with one or more other machines, either directly over a point-to-point communication path, over a wide area network (WAN) (e.g., the Internet), over a local area network (LAN), over a cellular network, or over another communication means. Further, the communication port 224 may communicate with an antenna or other link for electromagnetic signal transmission and/or reception.

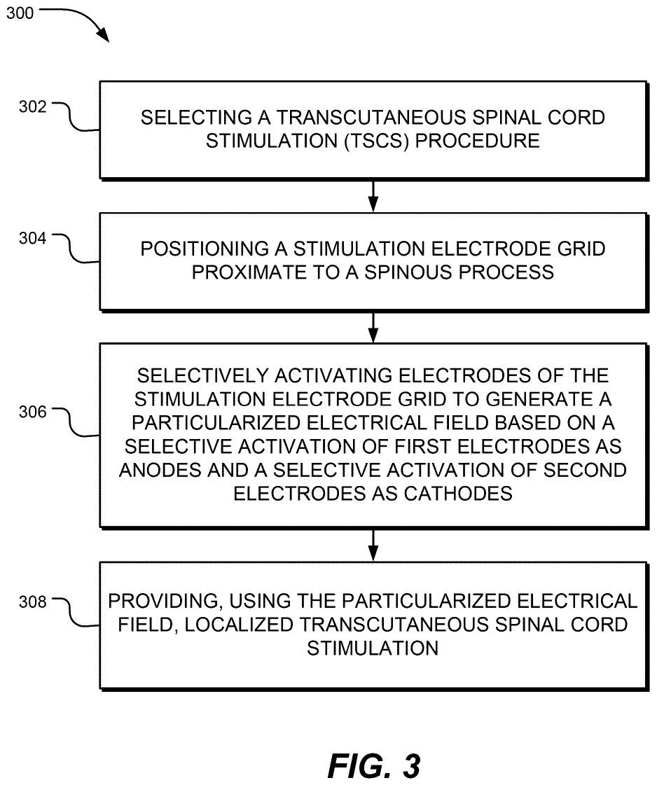

FIG. 3 depicts example method(s) 300 of tSCS which can be performed by any of the system(s) 100 disclosed herein.

At operation 302, the method 300 can select a tSCS procedure. At operation 304, the method 300 can position a stimulation electrode grid proximate to a spinous process. At operation 306, the method 300 can selectively activate electrodes of the stimulation electrode grid to generate a particularized electrical field based on a selective activation of first electrodes as anodes and a selective activation of second electrodes as cathodes. The selectively activating of the electrodes can include receiving a user input at a graphical user interface of a mobile device associated with a user, the user input selecting an electrode grid activation configuration from a plurality of electrode grid activation configurations presented at the graphical user interface. At operation 106, the method 300 can provide, using the particularized electrical field, localized tSCS. In some scenarios, the localized tSCS can include a plurality of 0.1 ms biphasic pulses delivered at 100 Hz. Additionally or alternatively, the localized tSCS can use 40 mA distributed across eight channels corresponding to eight electrodes, in parallel, resulting in a delivery of 5 mA per channel.

In some examples, the localized current stimulation provided by the system(s) 100 disclosed herein can use a smaller electrode diameter which can enhance the selectivity. Thus, the tSCS procedure 102 can enhance the stimulation-induced effect using a localized stimulation approach by employing a spatially confined grid of electrodes design that includes both the anodes 122 and cathodes 124, integrated into a single electrode grid unit (e.g., being disposed on the same piece of substrate material 110). A simulation study, using a simulation of a high-fidelity human spinal cord model. can show that the tSCS procedure 102 can generate an electric field strong enough to reach the spinal cord through

In some instances, the system(s) 100 disclosed herein can deliver biphasic pulses at 100 Hz and can administer 40 mA stimulation for 20 minutes. Other implementations can modulate lower motor functions primarily applied biphasic pulses with frequencies ranging from 1 to 90 Hz, and the stimulation duration in these examples can vary between 5 and 45 minutes.

In some instances, the system(s) 100 can use spinal cord maps of motoneuron pool for the anterior shank muscle (e.g., TA; L4-L5 spinal cord segments), which can be segmentally separate from the posterior shank muscles (e.g., Gastrocnemius, Sol; S1-S2) with minimal overlap.

In some instances, given the inherent variability of the anatomical structures and the neurophysiology of spinal circuits across individuals. Factors such as the distance between the spinal cord and skin, as well as the volume and thickness of other underlying structures, can influence the resulting electric field generated by the spinal stimulation system in each participant. Individualizing the stimulation intensity for each participant may provide a more precise understanding of the stimulation effects. The system(s) 100 can employ a systematic method, setting stimulation intensities from 1.5 to 210 mA. The optimal intensity can then be chosen by raising the intensities in increments of 1 to 5 mA up to the tolerance capacity of each participant. Additionally or alternatively, a fixed level of electric current can be used across subjects. Determining the individualized optimal intensity of the stimulation may, in some examples, provide more reliable results among users.

Furthermore, the system(s) 100 can benefit patients by eliminating the need for surgical procedures and their associated recovery period. The guided noninvasive spinal cord stimulation design disclosed herein can be a viable tool to enhance outcomes in targeted motor training or rehabilitation scenarios.

It is to be understood that the specific order or hierarchy of steps in the methods depicted throughout this disclosure are instances of example approaches and can be rearranged while remaining within the disclosed subject matter. For instance, any of the operations discussed throughout this disclosure may be omitted, repeated, performed in parallel, performed in a different order, and/or combined with any other of the operations discussed throughout this disclosure.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. Any component or feature of one example disclosed herein can be combined with any component or feature of any other example disclosed herein. These and other variations, modifications, additions, and improvements may fall within the scope of the disclosure as defined in the claims that follow.

What is claimed is:

1. A system of transcutaneous spinal cord stimulation (tSCS), the system comprising:
a stimulation electrode grid including a plurality of anodes and a plurality of cathodes, the stimulation electrode grid operable for placement proximate to a spinous process;
a particularized electrical field generated by selective activation of the stimulation electrode grid; and
a localized tSCS, provided by the particularized electrical field, and operable to cause a neuromodulation effect.

2. The system of claim 1,
wherein,
the stimulation electrode grid includes a substrate material, and
the plurality of cathodes and the plurality of anodes are both arranged as rows or columns on the substrate material to form an integrated unit.

3. The system of claim 1,
wherein,
the stimulation electrode grid includes an electrode array activation configuration with:
the plurality of anodes forming a first outer column of electrodes and a second outer column of electrodes, and
the plurality of cathodes forming a first inner column of electrodes and a second inner column of electrodes.

4. The system of claim 1,
wherein,
the stimulation electrode grid includes at least a four-by-four electrode array with:
the plurality of anodes including four electrodes located in a quadrant I of the four-by-four electrode array, and
the plurality of cathodes including four electrodes located in a quadrant III of the four-by-four electrode array.

5. The system of claim 1,
wherein,
the stimulation electrode grid includes an electrode array activation configuration with:
the plurality of anodes forming a first outer column of electrodes and a first inner column of electrodes, and
the plurality of cathodes forming a second outer column of electrodes and a second inner column of electrodes.

6. The system of claim 1,
wherein,
the stimulation electrode grid includes an electrode array activation configuration with:
the plurality of anodes forming a first two-by-two electrode array, and
the plurality of cathodes forming a second two-by-two electrode array adjacent to the first two-by-two electrode array.

7. The system of claim 1, further comprising:
wherein,
the stimulation electrode grid includes an electrode array rotated 45° relative to a longitudinal spinal axis.

8. The system of claim 1, further comprising:
a current generator with a plurality of output channels operable to provide a current signal to the stimulation electrode grid, responsive to a selective activation, which causes the particularized electrical field.

9. The system of claim 8, further comprising:
a mobile computing device communicatively coupled to the current generator, and the selective activation is in response to a user input at a graphical user interface of the mobile computing device.

10. A transcutaneous spinal cord stimulation device comprising:
a substrate material;
a stimulation electrode grid, disposed on the substrate material, and including a plurality of anodes and a plurality of cathodes, the stimulation electrode grid operable for placement proximate to a spinous process;
a power supply input; and
a current generator, coupled to the power supply input, and including a plurality of output channels operable to provide a current signal to the stimulation electrode grid resulting in generation of a particularized electrical field, the particularized electrical field provides localized transcutaneous spinal cord stimulation (tSCS).

11. The device of claim 10,
wherein,
the stimulation electrode grid includes a four-by-four electrode array selectable between a plurality of electrode array activation configurations to provide a plurality of different particularized electrical fields.

12. The device of claim 11,
wherein,
the plurality of electrode array activation configurations are selectively activated and include:
a first electrode array activation configuration with outer columns of electrodes activated to be the plurality of anodes and inner columns of electrodes activated to be the plurality of cathodes, and
a second electrode array activation configuration with electrodes at a first side activated to be the plurality of anodes and electrodes at a second side, opposite the first side, activated to be the plurality of cathodes.

13. The device of claim 11,
wherein,
the plurality of electrode array activation configurations include:
a first electrode array activation configuration with electrodes at a first quadrant activated to be the plurality of anodes and electrodes at a second quadrant activated to be the plurality of cathodes, and
a second electrode array activation configuration with electrodes at a first side activated to be the plurality of anodes and electrodes at a second side, opposite the first side, activated to be the plurality of cathodes.

14. The device of claim 11,
wherein,
the plurality of electrode array activation configurations includes an electrode array activation configuration with a first two-by-two array of electrodes activated to be the plurality of anodes and a second two-by-two array of electrodes, adjacent to the first two-by-two array, activated to be the plurality of cathodes.

15. The device of claim 14,
wherein,
the electrode array activation configuration includes:
a first row of inactive electrodes above the plurality of anodes and the plurality of cathodes, and
a second row of inactive electrodes below the plurality of anodes and the plurality of cathodes.

16. The device of claim 10,
wherein,
the localized tSCS provides at least one of:
a neuromodulation of a spinal reflex excitability, or
a neuromodulation for pain management.

17. The device of claim 14, further comprising:
wherein,
the particularized electrical field targets one or more lumbar spinal circuits which innervates a limb muscle.

18. The device of claim 10,
wherein,
the stimulation electrode grid includes a grid layout with:
an equal distance between electrodes, and
a number of cathodes equaling a number of anodes.

19. The device of claim 10, further comprising:
a positioning guide operable to assist in placement of the stimulation electrode grid.

20. The device of claim 10, further comprising:
a plurality of predetermined stimulation modes stored in a memory of a computing device communicatively coupled to the stimulation electrode grid, the plurality of predetermined stimulation modes corresponding to different target diseases.

21. A method of transcutaneous spinal cord stimulation (tSCS) comprising:
selecting a tSCS procedure;
positioning a stimulation electrode grid proximate to a spinous process;
selectively activating electrodes of the stimulation electrode grid to generate a particularized electrical field based on a selective activation which assigns first electrodes as anodes and a selective activation which assigns second electrodes as cathodes; and
providing, using the particularized electrical field, localized transcutaneous spinal cord stimulation (tSCS).

22. The method of claim 21,
wherein,
the localized tSCS includes a plurality of 0.1 ms biphasic pulses delivered at 100 Hz, or
the localized tSCS uses 40 mA distributed across eight channels corresponding to eight electrodes, in parallel, resulting in a delivery of 5 mA per channel.

23. The method of claim 21,
wherein,
the selective activating of the electrodes includes receiving a user input at a graphical user interface of a mobile device associated with a user, the user input selecting an electrode grid activation configuration from a plurality of electrode grid activation configurations presented at the graphical user interface.

24. The method of claim 21,
wherein,
the localized transcutaneous spinal cord stimulation modulates a spinal reflex excitability corresponding to extremities of a treatment recipient.

25. The method of claim 21,
providing data representing the selective activation to a machine-learning model trained to identify patterns of use of different selective activations.
